SEQUENCE LISTING (10 sequences)

SEQ ID NO: 1 - DNA, 20 bp, Artificial Sequence
  sense primer
agaccatgtttctgaagtgc

SEQ ID NO: 2 - DNA, 19 bp, Artificial Sequence
  antisense primer
gaaccatactcgaaccaca

SEQ ID NO: 3 - DNA, 20 bp, Artificial Sequence
  sense primer
ctagttctgaggaagcatcg

SEQ ID NO: 4 - DNA, 19 bp, Artificial Sequence
  antisense primer
tctcagtagcagacccttg

SEQ ID NO: 5 - DNA, 19 bp, Artificial Sequence
  sense primer
aactctggcgatgggtgtt

SEQ ID NO: 6 - DNA, 19 bp, Artificial Sequence
  antisense primer
aaactggaagggtgggaca

SEQ ID NO: 7 - DNA, 20 bp, Artificial Sequence
  sense primer
cgtgtgtgatgtgaggtgag

SEQ ID NO: 8 - DNA, 17 bp, Artificial Sequence
  antisense  primer
ctccgtggctggtttca

SEQ ID NO: 9 - DNA, 20 bp, Artificial Sequence
  sense primer
ttttcgtctctagccgcgtg

SEQ ID NO: 10 - DNA, 21 bp, Artificial Sequence
  antisense primer
ggccctgggcacatacttgtg

(19) United States
(12) Patent Application Publication
(10) Pub. No.: US 2014/0170746 A1
(43) Pub. Date: Jun. 19, 2014

(54) METHOD FOR PREPARING INDUCED PLURIPOTENT STEM CELLS USING MICROVESICLES DERIVED FROM EMBRYONIC STEM CELLS

(71) Applicants: Jae-Sung Park, Gyeongsangbuk-do (KR); Yong Song Gho, Gyeongsangbuk-do (KR); Yoon Keun Kim, Gyeongsangbuk-do (KR); Jun Ho Kim, Gyeongsangbuk-do (KR); Su Chul Jang, Gyeongsangbuk-do (KR); Namwoo Yi, Seoul (KR); Dayeong Jeong, Ulsan (KR); Eun-Jeong Choi, Busan (KR)

(72) Inventors: Jae-Sung Park, Gyeongsangbuk-do (KR); Yong Song Gho, Gyeongsangbuk-do (KR); Yoon Keun Kim, Gyeongsangbuk-do (KR); Jun Ho Kim, Gyeongsangbuk-do (KR); Su Chul Jang, Gyeongsangbuk-do (KR); Namwoo Yi, Seoul (KR); Dayeong Jeong, Ulsan (KR); Eun-Jeong Choi, Busan (KR)

(73) Assignee: POSTECH ACADEMY-INDUSTRY FOUNDATION, Gyeongsangbuk-do (KR)

(21) Appl. No.: 14/114,190

(22) PCT Filed: Apr. 19, 2012
(86) PCT No.: PCT/KR12/03019

§ 371 Date: Feb. 7, 2014

(30) Foreign Application Priority Data
Apr. 28, 2011 (KR) 10-2011-0040202

(51) Int. Cl. C12N 5/074

(52) U.S. Cl. CPC C12N 5/0696
USPC 435/326; 435/350; 435/351; 435/353; 435/354; 435/363; 435/366; 435/377

(57) ABSTRACT

Provided is a method of dedifferentiating somatic cells using embryonic stem cell-derived microvesicles. Particularly, a method of preparing induced pluripotent stem cells by treating a composition including embryonic stem cell-derived microvesicles to the somatic cells. According to the method of preparing induced pluripotent stem cells, the dedifferentiation of the somatic cells may be efficiently performed without side effects using the embryonic stem cell-derived microvesicles, and moreover, the method is expected to be very useful in developing a cell therapy product having immunocompatibilities by individuals.

### TECHNICAL FIELD

The present invention relates to a method of preparing induced pluripotent stem cells by dedifferentiating somatic cells using embryonic stem cell-derived microvesicles.

### BACKGROUND ART

Dedifferentiation is a process in which mature somatic cells revert to younger stem cells, and relates to regeneration in vivo, which occurs in insects, amphibians, plants. It does not occur naturally in mammals such as humans, but only through artificial methods. Cellular dedifferentiation methods began with methods using cell fusion. The first method of dedifferentiating somatic cells to be discovered was a method using a characteristic in which, when embryonic stem cells (ESCs) are fused with somatic cells, the embryonic stem cells become more dominant. Afterward, research on inducing dedifferentiation of the somatic cells has progressed through fusion of somatic cells with stem cells having similar capabilities to the ESCs, for example, embryonic germinal cells (EGCs), or embryonic carcinoma cells (ECCs) in addition to the ESCs.

However, cells in which cell fusion occurs are randomly divided or maintained in a fused state without division. While a cell in which division occurs (hybrid) has one nucleus, a cell in which division does not occur (heterokaryon) has nuclei of two different cells. For this reason, most cells become cancer cells, and only some cells have normal functions.

In addition, the method of examining cell fusion (cell, cytoplasm) has a disadvantage in that many cells die due to induction of fusion using polyethylene glycol (PEG) that can damage the cells to help fusion of large-sized cells. To solve this problem, conventional research on dedifferentiating only parts of cells rather than entire embryonic stem cells has progressed. One research was performed to exclude a nucleus from fusion by isolating only cytoplasm from a cell. A method of treating somatic cells with a chemical material (streptolysin) such that a cell membrane has permeability, and inserting cytoplasm derived from embryonic stem cells was used. However, in the case of the dedifferentiation using cytoplasm, there has been no successful case of dedifferentiation having similar characteristics to embryonic stem cells as of 2010.

In addition, there is a delivery system of a specific factor. In 2006, the Yamanaka research team of Tokyo University in Japan revealed that dedifferentiation can be performed with only four genes in embryonic stem cells. The four genes (Oct-4, klf4, Sox2, and c-Myc) were introduced into mouse skin cells, thereby preparing induced pluripotent stem cells (iPSs). This is research that began with a concept in which a part of a cell is synthesized for insertion, rather than fusion of a whole cell. The iPS technique used a method of producing a protein by inserting genes into a genome using a capability of a virus in an initial stage and compulsorily expressing RNA. However, due to the characteristics of a virus, the genes are integrated in several sites, thereby forming undesired modifications. Today, to solve this problem, an mRNA delivery method, or a method of delivery a protein itself is used. However, it was seen that mRNA is easily degraded and has a difficult synthesis process, an immune response of RNA is induced, and the delivery of only a protein cannot achieve perfect dedifferentiation.

Accordingly, there is a demand for research on a method of preparing dedifferentiated pluripotent stem cells that can overcome the above-described problems.

### DISCLOSURE

### Technical Problem

As a result of research to solve the problems according to the conventional art, the inventors found that dedifferentiation of somatic cells can be effectively and stably performed using embryonic stem cell-derived microvesicles.

Accordingly, the present invention is directed to providing a method of preparing induced pluripotent stem cells by efficiently performing dedifferentiation of somatic cells with no side effects using embryonic stem cell-derived microvesicles, and an induced pluripotent stem cell prepared thereby.

However, the technical problem is not limited to the above description, and other problems not to be described will be clearly understood by one of ordinary skill in the art with respect to descriptions below.

### Technical Solution

One aspect of the present invention provides a method of preparing induced pluripotent stem cells, which includes treating a composition including embryonic stem cell-derived microvesicles to somatic cells.

Another aspect of the present invention provides induced pluripotent stem cells prepared by treating a composition including embryonic stem cell-derived microvesicles to somatic cells.

Still another aspect of the present invention provides a cell therapy product including the induced pluripotent stem cells.

### Advantageous Effects

According to the present invention, a cytoplasm delivery method using fusion of microvesicles does not need polyethyleneglycol (PEG) needed in cell fusion or a cytotoxic material such as streptolysin used in cytoplasm injection, and thus cells are damaged less. In addition, during the cytoplasm delivery, the microvesicle has high delivery efficiency, and more effectively protects a delivered content, and thus it is possible to perform specific delivery of the cytoplasm. According to conventional research on cytoplasm delivery, cytoplasm delivery efficiency is decreased according to an amount of proteins to be expressed in a nucleus, and thus perfect dedifferentiation does not occur. However, the microvesicles of the present invention can freely control a concentration of the cytoplasm and prevent loss of intracellular materials in fusion depending on a preparation method, so that the efficiency of dedifferentiation of somatic cells using the cytoplasm can be increased.

In addition, according to the method using microvesicles of the present invention, targeting to induce in-vivo dedifferentiation is possible. Particularly, since a signal generated in a wounded organ of a patient is a major factor of targeting, the dedifferentiation performed in vitro is expected to become a new means capable of being performed in vivo.

### DESCRIPTION OF DRAWINGS

FIG. 1 shows transmission electron microscope (TEM) images of embryonic stem cell-derived microvesicles prepared by extrusion.

FIG. 2 is a graph showing a size of an embryonic stem cell-derived microvesicle prepared by extrusion.

FIG. 3 is a diagram showing that an embryonic stem cell-specific protein, that is, Oct3/ 4, is present in embryonic stem cells and embryonic stem cell-derived microvesicles.

FIG. 4 is a diagram showing that embryonic stem cell-specific genes, that is, Oct3/4 and Nanog, are present in embryonic stem cells and embryonic stem cell-derived microvesicles.

FIG. 5 shows colonies produced after dedifferentiation of mouse fibroblasts (NIH3T3 cells) using embryonic stem cell-derived microvesicles.

FIG. 6 shows that colonies produced after mouse fibroblasts (NIH3T3 cells) are dedifferentiated using embryonic stem cell-derived microvesicles and proliferated by lapse of time.

FIG. 7 shows colonies produced after GFP-transformed mouse fibroblasts (NIH3T3-GFP cells) are dedifferentiated using embryonic stem cell-derived microvesicles.

FIG. 8 is a diagram showing that embryonic stem cell-specific genes, that is, Oct3/4 and Nanog, are expressed in dedifferentiated mouse fibroblasts (NIH3T3 dedifferentiated cells).

FIG. 9 shows that dedifferentiated mouse fibroblasts (NIH3T3 dedifferentiated cells) have differentiation capability.

FIG. 10 is a diagram showing that differentiation-specific genes, that is, AFP, Foxf1, and β-III tubulin, are expressed when dedifferentiated mouse fibroblasts (NIH3T3 dedifferentiated cells) are differentiated.

FIG. 11 shows colonies produced after mouse fibroblasts (NIH3T3 cells) are dedifferentiated by treating embryonic stem cell-derived microvesicles in combination with brefeldin A (BFA).

### MODES OF INVENTION

The present invention provides a method of preparing induced pluripotent stem cells by treating a composition including embryonic stem cell-derived microvesicles to somatic cells to dedifferentiate the somatic cells.

The embryonic stem cells used in the present invention may be derived from humans, non-human primates, mice, rats, dogs, cats, horses, and cattle, but the present invention is not limited thereto.

The “microvesicle” used herein is divided into internal and external sides by a lipid bilayer composed of a cell membrane component of a derived cell, includes a cell membrane lipid, a cell membrane protein, a nucleic acid and cell components of the cell, and has a smaller size than the original one, but the present invention is not limited thereto.

The microvesicle of the present invention may be prepared of a suspension including embryonic stem cells by a method selected from the group consisting of extrusion, sonication, cytolysis, homogenization, freezing-defrosting, electroporation, mechanical degradation, and treatment with a chemical material, but the present invention is not limited thereto.

In one embodiment of the present invention, a membrane of the microvesicle may further include a component other than the cell membrane of the embryonic stem cell.

The component other than the cell membrane may include a targeting molecule, a material necessary for fusion of the cell membrane with a targeting cell (fusogen), cyclodextrin, PEG, etc. In addition, the component other than the cell membrane may be added by various methods, which include chemical modification of the cell membrane.

For example, the membrane component of the microvesicle may be chemically modified by a chemical method using a thio group (—SH) or an amine group (—NH₂), or by chemically binding PEG to the microvesicle.

The present invention may further include chemically modifying the membrane component of the microvesicle in the preparation of the microvesicle of the present invention.

In addition, the embryonic stem cell of the present invention includes a transformed cell. Specifically, the transformed cell includes a cell transformed to express a material necessary for fusion of a cell membrane with a specific protein, a targeting molecule, or a target cell; and a transformed cell composed of a combination of at least two thereof, but the present invention is not limited thereto.

The embryonic stem cell may be transformed by treatment of a material or transduction, and may be transformed at least twice.

In another embodiment of the present invention, the embryonic stem cell may be transformed to inhibit expression of at least one specific protein.

In still another embodiment of the present invention, the embryonic stem cell may be transformed to express at least one selected from the group consisting of a cell adhesion molecule, an antibody, a targeting protein, a cell membrane fusion protein, and a fusion protein thereof.

In yet another embodiment of the present invention, the embryonic stem cells may be transformed to overexpress an embryonic stem cell-specific protein, that is, Oct3/4, Nanog, or Sox-2 protein.

In yet another embodiment of the present invention, the composition of the present invention may further include a component other than the embryonic stem cell-derived microvesicle.

For example, the composition including the embryonic stem cell-derived microvesicle and a material stimulating dedifferentiation of somatic cells may be treated to the somatic cells. The additional material includes brefeldin A (BFA), BiP inducer X (BIX), and valproic acid (VPA).

Hereinafter, exemplary Examples are provided to help understanding of the present invention. However, the following Examples are provided that the present invention may be more easily understood, and the scope of the present invention is not limited to Examples.

### EXAMPLE 1

### Preparation of Embryonic Stem Cell-Derived Microvesicles

Mouse embryonic stem cells were resuspended in 3 ml of a phosphate buffered saline (PBS) solution at a concentration of 5×10⁶ cells/ml. The resuspension was passed through a membrane filter having a pore size of 10 μm 10 times, and through a membrane filter having a pore size of 5 μm 10 times. 1 ml of 50% OptiPrep™, 1 ml of 5% OptiPrep™, and 3 ml of a cell suspension passed through the membrane filter were each put in a 5 ml ultracentrifuge tube. Afterward, ultracentrifugation was performed at 100,000×g for 2 hours. A microvesicle was obtained from a layer between 50% OptiPrep™ and 5% OptiPrep™.

### EXAMPLE 2

### Analysis of Characteristics of Embryonic Stem Cell-Derived Microvesicles

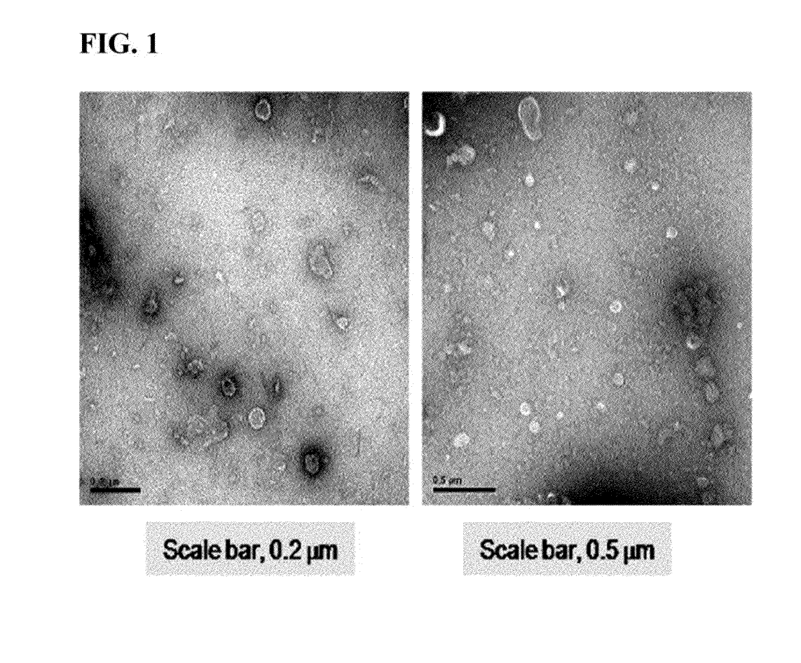

The microvesicles prepared in the embryonic stem cells according to the method described in Example 1 were adsorbed on a glow-discharged carbon-coated copper grid for 3 minutes. The grid was washed with distilled water and stained with 2% uranylacetate for 1 minute, and results observed using a transmission electron microscope, JEM101 (Jeol, Japan), are shown in FIG. 1.

As shown in the TEM images of FIG. 1, it can be seen that the microvesicle prepared by extrusion from the embryonic stem cells was composed of a lipid bilayer, and usually formed in a sphere having a size of 100 to 200 nm.

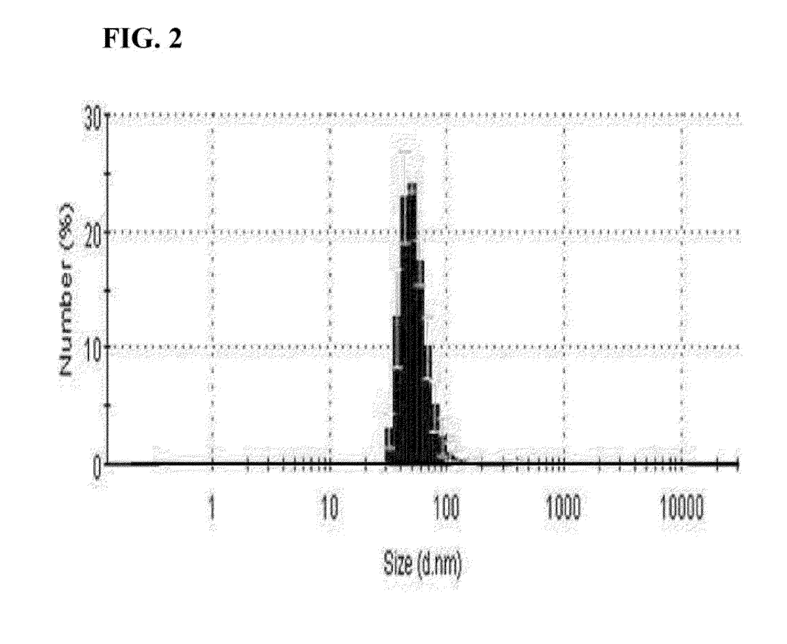

The microvesicle prepared from the embryonic stem cells described in Example 1 was diluted in 1 ml of PBS at a concentration of 5 μg/ml. 1 ml of PBS containing the microvesicles was put into a cuvette and analyzed using a dynamic light scattering particle size analyzer, and results are shown in FIG. 2.

As shown in FIG. 2, it is confirmed that the microvesicle had a size of 50 to 100 nm, and an average size of 70 nm

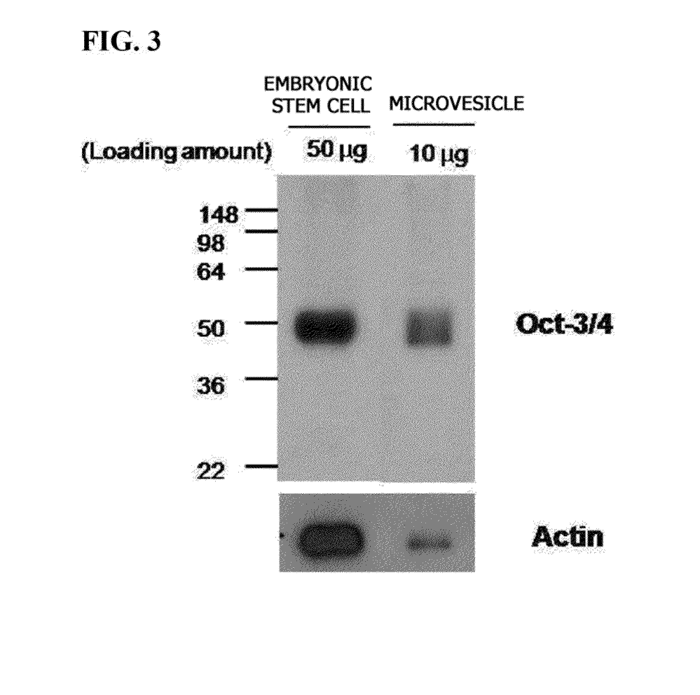

50 μg of the microvesicles prepared in the embryonic stem cells according to the method described in Example 1 and 10 μg of a whole cell lysate of the embryonic stem cells were prepared, a 5× loading dye (250 mM Tris-HCl, 10% SDS, 0.5% bromophenol blue, 50% glycerol) was added to finally become 1×, and the resulting solution was treated at 100° C. for 5 minutes. 8% polyacrylamide gel was prepared, and then a sample was loaded. The sample was subjected to electrophoresis at 80 V for 2 hours, and a protein was transferred to a polyvinylidene fluoride (PVDF) membrane at 400 mA for 2 hours. Skim milk was dissolved in PBS to have a concentration of 3%, and the membrane was blocked in the solution for 2 hours. Oct3/4 and β-actin antibodies were treated at 4° C. for 12 hours. The resulting membrane was washed with PBS twice, and secondary antibodies to which a peroxidase was attached were treated at room temperature for 1 hour. The resulting membrane was washed with PBS for 30 minutes and identified using an enhanced chemiluminescence (ECL; Amersham Co. No. RPN2106) substrate, results of which are shown in FIG. 3.

As shown in FIG. 3, it is confirmed that an embryonic stem cell-specific protein, that is, Oct3/4, was present in the microvesicles prepared in the embryonic stem cells.

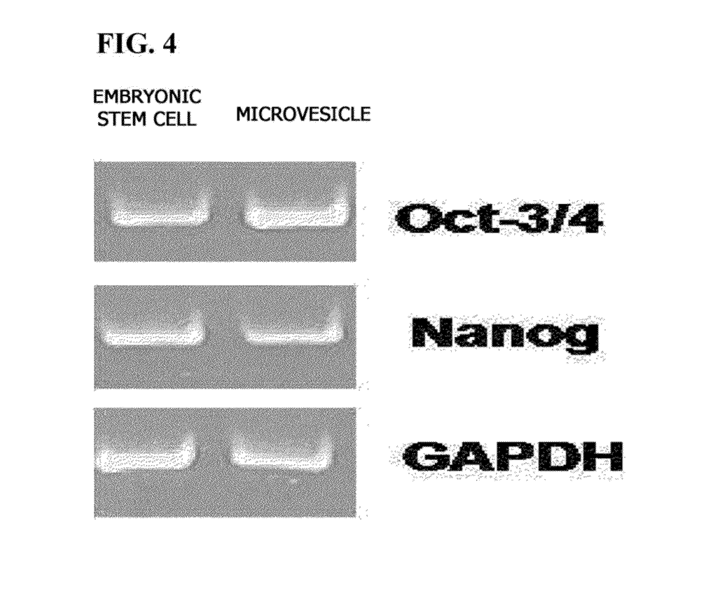

Total genes (RNAs) were extracted from the microvesicles prepared in the embryonic stem cells according to the method described in Example 1 and the embryonic stem cells using an RNeasy® Mini kit (QIAGEN, Cat. No. 74104). A plurality of cDNAs were obtained from the extracted RNA using a specific gene primer and a PCR kit (BIOLAB, Cat. No. E5000), isolated by agarose gel electrophoresis, and identified using ethidium bromide (ETBR) staining. To confirm that the same amount of RNAs as a positive control (embryonic stem cells) was used, a housekeeping gene, glyceraldehyde 3-phosphate dehydrogenase (GAPDH), was identified together. Here, as a sense primer of an embryonic stem cell-specific gene, that is, Oct-3/4, 5′-AGACCATGTTTCTGAAGTGC-3′ was used, and as an antisense primer thereof, 5′-GAACCATACTCGAACCACA-3′, was used. As a sense primer of another embryonic stem cell-specific gene, that is, Nanog, 5′-CTAGTTCTGAGGAAGCATCG-3′ was used, and as an antisense primer thereof, 5′-TCTCAGTAGCAGACCCTTG-3′ was used. For analysis of characteristics of the embryonic stem cell-derived microvesicles, gene expression images are shown in FIG. 4.

As shown in FIG. 4, it is confirmed that embryonic stem cell-specific genes, that is, Oct3/4 and Nanog, were expressed in the microvesicles prepared in the embryonic stem cells.

### EXAMPLE 3

### Dedifferentiation of Somatic Cells using Embryonic Stem Cell-Derived Microvesicles

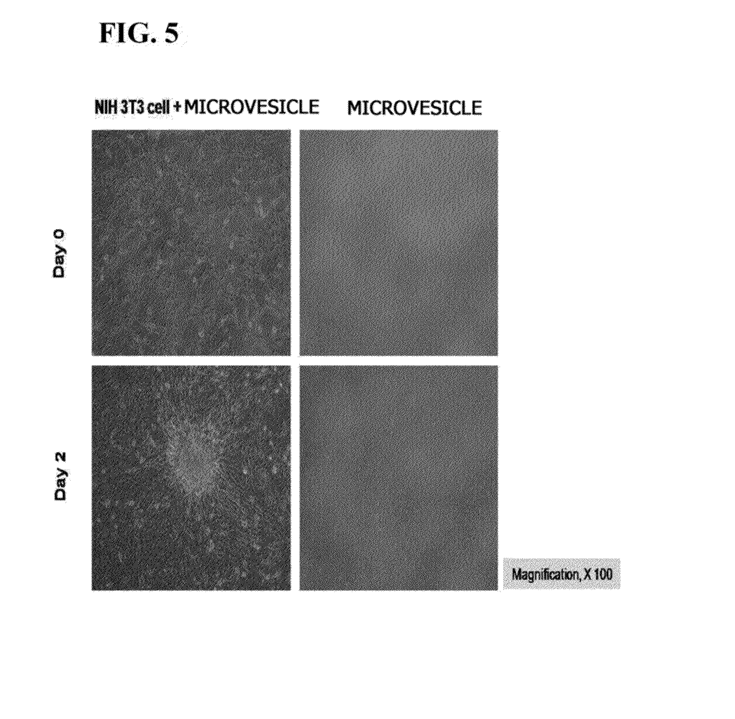

0.1% gelatin was coated on a 6-well plate and inoculated with 8×10⁴ of NIH3T3 cells, and the cells were incubated for 24 hours. Afterward, each well was washed with PBS, 2 ml of the microvesicles prepared in the embryonic stem cells according to the method described in Example 1 were diluted in a fibroblast medium (DMEM, 10% FBS, 100 U/ml penicillin-streptomycin) at a concentration of 100 μg/ml, and then treated to the incubated NIH3T3 cells. After 48 hours, approximately 2 to 3 colonies per well were identified, and each colony had a size of approximately 10 to 100 μm. The colonies were observed using an electron microscope, and results are shown in FIG. 5.

As shown in FIG. 5, it is confirmed that the NIH3T3 cells were dedifferentiated using the embryonic stem cell-derived microvesicles, thereby inducing colonies. Each well was washed with PBS, and 400 μl of 0.1× TE (Typsin-EDTA) was added. After 1 minute, 10 μl of 1× TE was added to the colony using a pipette, and sucked up by placing a tip of the pipette to surround the colony. The pick-up colonies were diluted in 500 μl of an embryonic stem cell medium (knock-out DMEM, 15% knock-out FBS, 10 ng/ml LIF, 0.1 mM 2-mercaptoethanol, 4 mM L-glutamine, 10 μg/ml of gentamycin, 100 U/ml penicillin-streptomycin), inoculated into a 0.1% gelatin-coated 24-well plate, and incubated for 5 days or more.

At the point of time at which the cells had grown to 80% confluency or more, whole somatic cells were diluted in 2 ml of the embryonic stem cell medium using 1× TE, and subcultured in a 0.1% gelatin-coated 6-well plate. Through the same method as described above, at the point of time at which the cells had grown to 80% confluency, the cells were diluted in 8 ml of a medium, subcultured in a 0.1% gelatin-coated 100 mm culture dish, and subcultured again every 2 to 3 days.

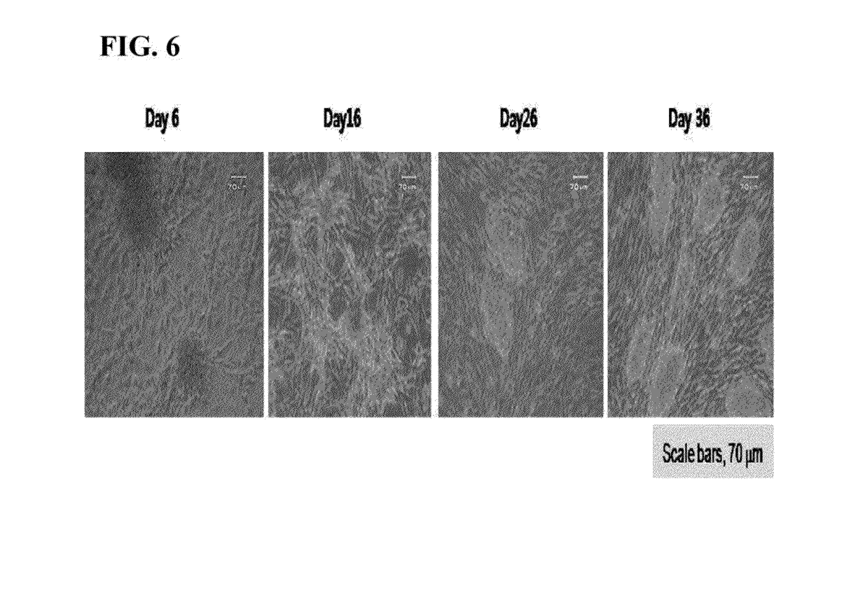

As shown in FIG. 6, it is confirmed from 5 or more days of observation that colonies were continuously proliferated.

### EXAMPLE 4

### Confirmation of Origin of Induced Pluripotent Stem Cells

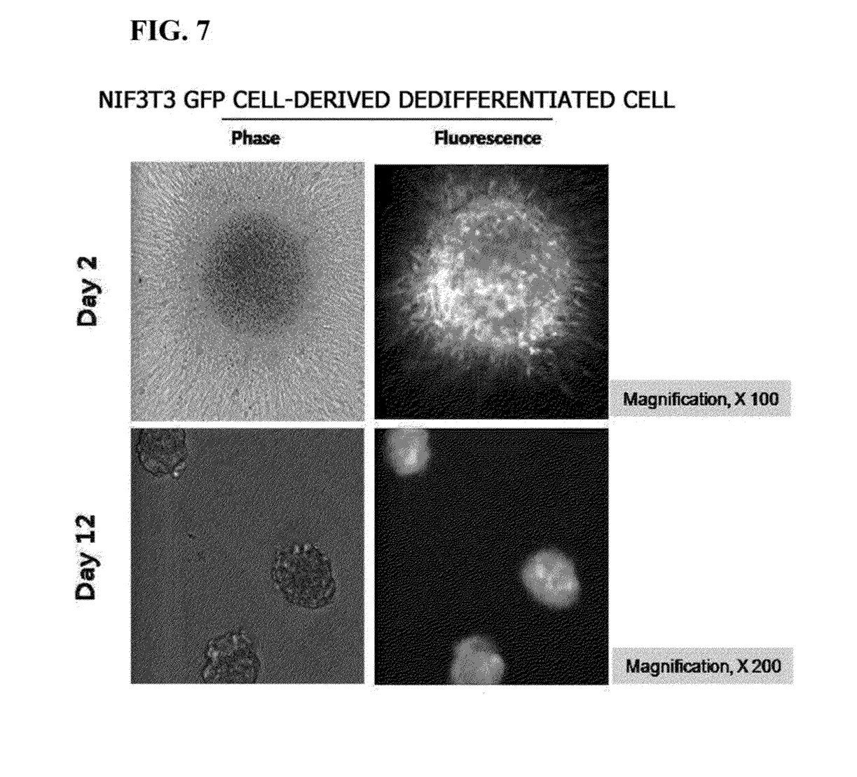

A GFP gene was injected into a genome of an NIH3T3 cell using a retrovirus (pMSCV). The transformed cell exhibited green fluorescence, but embryonic stem cells forming microvesicles did not exhibit fluorescence. A 6-well plate was coated with 0.1% gelatin and inoculated with 8×10⁴ of NIH3T3 GFP cells, and the cells were incubated for 24 hours. Afterward, each well was washed with PBS, and the embryonic stem cell-derived microvesicle prepared in Example 1 was diluted in 2 ml of a fibroblast medium (DMEM, 10% FBS, 100 U/ml penicillin-streptomycin) at a concentration of 100 μg/ml and treated to the NIH3T3 GFP cell. After 48 hours, approximately 2 to 3 colonies per well were identified, and a size of the colony was approximately 10 to 100 μm, and results observed by an electron microscope are shown in FIG. 7.

According to the conventional research, a long period, for example, at least 7 to 30 days, of dedifferentiation was needed, but, as shown in FIGS. 6 and 7, in the case of the embryonic stem cell-derived microvesicles of the present invention, it can be seen that colonies were formed in only 48 hours, and thus the period of dedifferentiation could be significantly reduced.

### EXAMPLE 5

### Confirmation of Characteristic of Induced Pluripotent Stem Cells

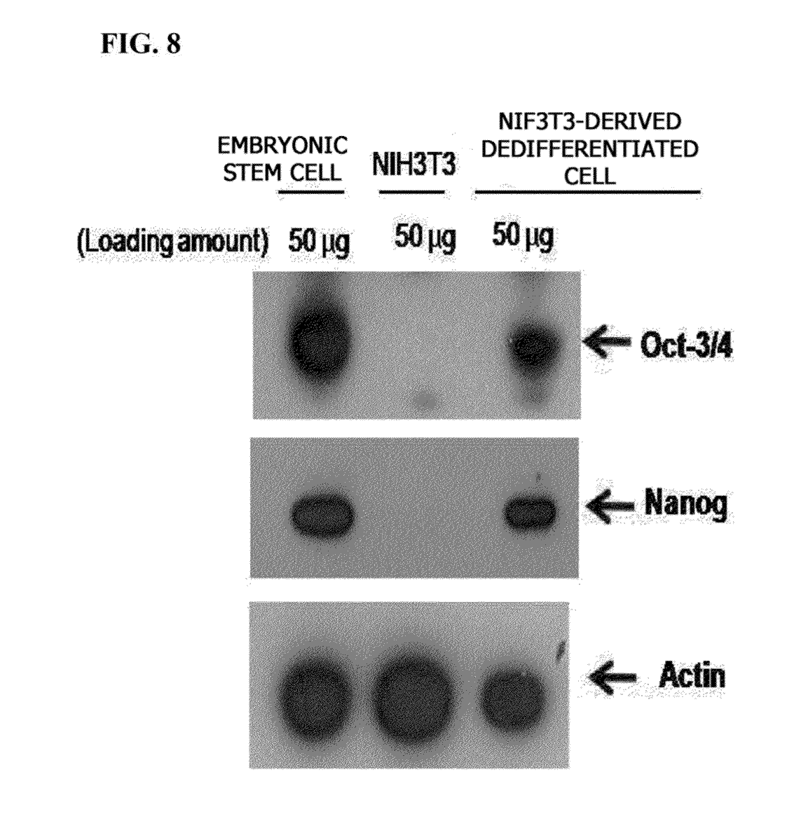

Colonies (NIH3T3-derived dedifferentiation cells) proliferated over 40 days according to the method described in Example 3, embryonic stem cells, and a whole cell lysate of the NIH3T3 cells were quantified by a detergent compatible protein assay (DC), and thus 50 μg of each was prepared, and a 5× loading dye was added to have a final concentration of 1×, and treated at 100° C. for 5 minutes. 8% polyamide gel was prepared, and a sample was loaded. The sample was subjected to electrophoresis at 80 V for 2 hours, and a protein was transferred to a PVDF membrane at 400 mA for 2 hours. The membrane used different blocking buffers according to antibodies to be applied. In the case of actin, skim milk was dissolved in PBS to have a concentration of 3%, and in the case of Oct 3/4, non fat dry milk was dissolved in PBS to have a concentration of 5%, in the case of Nanog, 10% non fat dry milk was dissolved in PBS to have a concentration of 10%, and the membrane was blocked in this solution for 2 hours. Oct3/4, Nanog, and β-actin antibodies were treated at 4° C. for 12 hours, the membrane was washed with PBS twice, and secondary antibodies to which a peroxidase was attached were treated at room temperature for 1 hour. The resulting membrane was washed with PBS for 30 minutes, and results were identified using an ECL substrate, which are shown in FIG. 8.

As shown in FIG. 8, it is confirmed that embryonic stem cell-specific proteins, that is, Oct3/4 and Nanog, were expressed in NIH3T3 dedifferentiation cells (induced pluripotent stem cells) dedifferentiated by embryonic stem cell-derived microvesicles.

### EXAMPLE 6

### Confirmation of Spontaneous Differentiation of Induced Pluripotent Stem Cells

To identify a function of the induced pluripotent cells proliferated over 40 days by the method described in Example 3, spontaneous differentiation capability to all cells was confirmed. When the embryonic stem cells became an embryonic body, the embryonic body had a capability to be spontaneously differentiated to all cells. By the same principle, cells induced by a hanging drop method were induced to compulsorily agglomerate. Colony cells were suspended using trypsin, and diluted in a differentiation medium (IMDM, 20% FBS, 4 mM L-glutamine, 10 μg/ml gentamycin, 100 U/ml penicillin-streptomycin) at a concentration of 3.3×10⁴/ml. 30 μl (1×10³) each of the diluted solution was attached to a top surface of a bacteria culture dish, and induced to agglomerate for 2 days. The cells agglomerating on the top surface were washed with a differentiation medium to be resuspended in a bacteria culture dish, and incubated for 2 days. FIG. 8 is images of agglomerated induced pluripotent stem cells suspended in the bacteria culture dish. By the same method, NIH3T3 cells and embryonic stem cells were induced to agglomerate by a hanging drop method.

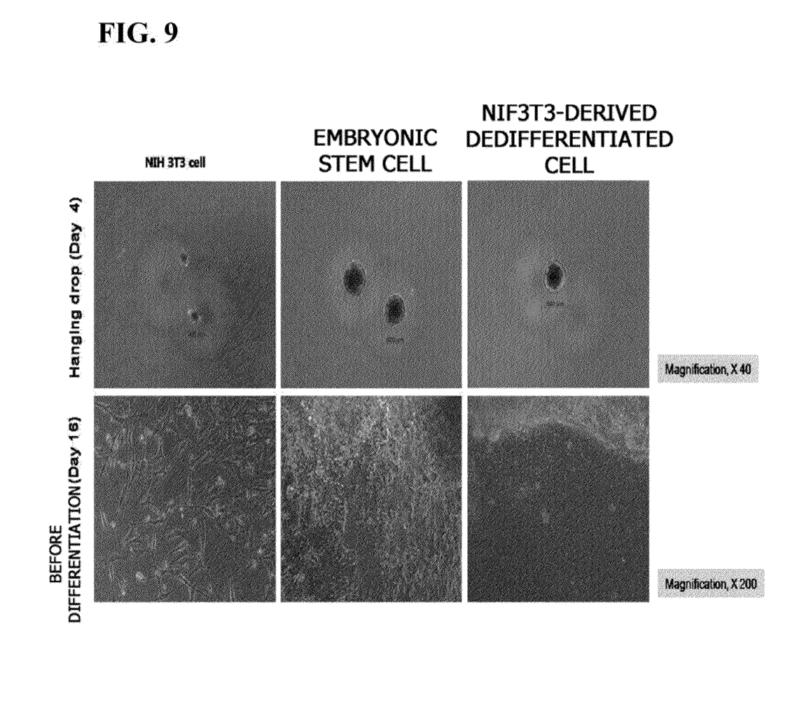

An embryonic body was diluted in a differentiation medium to have a confluency of 3/ml, and then 2 ml of the diluted body was injected into each well of a 0.1% gelatin-coated 6-well plate to incubate. A fresh culture solution was transferred twice, that is, every 8 days for 16 days. FIG. 9 shows the cells after induction to differentiation.

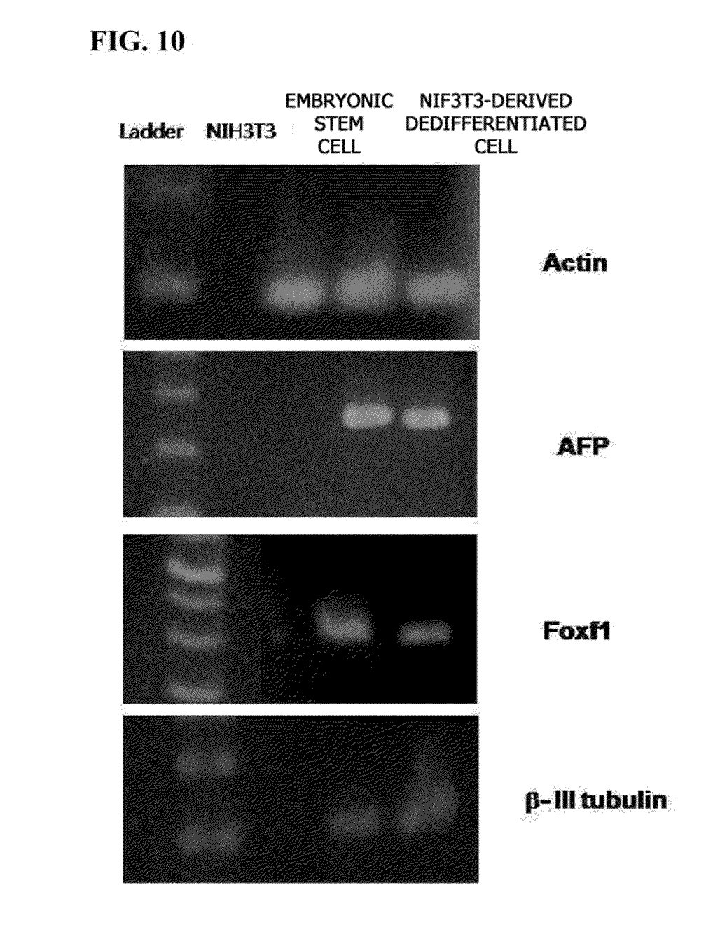

Total genes (RNAs) were extracted from the differentiated cells using an RNeasy® Mini kit (QIAGEN, Cat. No. 74104). A plurality of cDNAs were obtained from the extracted RNAs using a specific gene primer and a PCR kit (BIOLAB, Cat. No. E5000), isolated by agarose gel electrophoresis, and identified by ETBR staining. To confirm that the same amount of RNAs as a positive control (embryonic stem cells) was used, a housekeeping gene, actin, was identified together. As a result of confirming the spontaneous differentiation of the induced pluripotent stem cells, it was confirmed that the cells were differentiated into an endoderm (AFP), a mesoderm (Foxf1), and an ectoderm (b-III tubulin) of NIH3T3 dedifferentiation cells. For AFP, 5′-AACTCTGGCGATGGGTGTT-3′ was used as a sense primer and 5′-AAACTGGAAGGGTGGGACA-3′ was used as an antisense primer. For Foxf1, 5′-CGTGTGTGATGTGAGGTGAG-3′ was used as a sense primer and 5′-CTCCGTGGCTGGTTTCA-3′ was used as an antisense primer. For b-III tubulin, 5′-TTTTCGTCTCTAGCCGCGTG-3′ was used as a sense primer and 5′-GGCCCTGGGCACATACTTGTG-3′ was used as an antisense primer. FIG. 10 shows images for checking whether the gene is expressed to confirm a spontaneous differentiation capability of NIH3T3 dedifferentiation cells (induced pluripotent stem cells).

As shown in FIG. 10, it is confirmed that endodermal, mesodermal, and ectodermal genes were expressed in the NIH3T3 dedifferentiation cells, which shows that the NIH3T3 dedifferentiation cells have a spontaneous differentiation capability.

### EXAMPLE 7

### Dedifferentiation of Somatic Cells According to Simultaneous Treatment of Embryonic Stem Cell-Derived Microvesicles and BFA

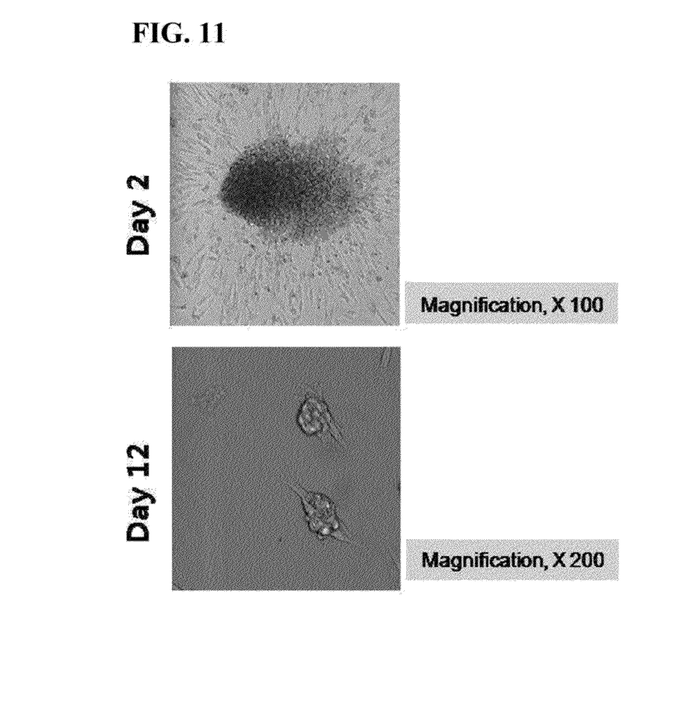

0.1% gelatin was coated on a 6-well plate, and 8×10⁴ of NIH3T3 cells were inoculated into each well and incubated for 24 hours. After the well was washed with PBS, the embryonic stem cell-derived microvesicles prepared in Example 1 were diluted in 2 ml of a fibroblast medium (DMEM, 10% FBS, 100 U/ml penicillin-streptomycin) at a concentration of 100 μg/ml, and BFA was diluted in 2 ml of a fibroblast medium (DMEM, 10% FBS, 100 U/ml penicillin-streptomycin) at a concentration of 2 μM, and then each of the resulting products was treated to the incubated NIH3T3 cells. After 48 hours, 2 to 3 colonies per well were identified, and a size of the colony was approximately 10 to 100 μm. Results observed by an electron microscope are shown in FIG. 11.

As shown in FIG. 11, it is confirmed that colonies could be induced even when embryonic stem cell-derived microvesicles and BFA were simultaneously treated.

Each well was washed with PBS, and 400 μl of 0.1× TE (Typsin-EDTA) was added. After 1 minute, 10 μl of 1× TE was added to the colony using a pipette, and sucked up by placing a tip of the pipette to surround the colony. The pick-up colonies were diluted in 500 μl of an embryonic stem cell medium (knock-out DMEM, 15% knock-out FBS, 10 ng/ml LIF, 0.1 mM 2-mercaptoethanol, 4 mM L-glutamine, 10 μg/ml gentamycin, 100 U/ml penicillin-streptomycin), inoculated into a 0.1% gelatin-coated 24-well plate, and incubated for 5 days or more.

As shown in FIG. 11, it is confirmed that the colonies were continuously proliferated.

While the invention has been shown and described with reference to certain exemplary embodiments thereof, it will be understood by those skilled in the art that various modifications in form and details may be made therein without departing from the spirit and scope of the invention as defined by the appended claims.

### INDUSTRIAL APPLICABILITY

Since a method of preparing induced pluripotent stem cells according to the present invention uses a cytoplasm delivery method using fusion of microvesicles, it is expected that dedifferentiation of somatic cells can be efficiently performed without side effects, and the method can be effectively used to develop a cell therapy product having different immunocompatibilities with individuals.

1. A method of preparing induced pluripotent stem cells, comprising:
treating a composition including embryonic stem cell-derived microvesicles to somatic cells.

2. The method of according to claim 1, wherein the embryonic stem cell is derived from one selected from the group consisting of a human, a non-human primate, a mouse, a rat, a dog, a cat, a horse, and cattle.

3. The method of claim 1, wherein the embryonic stem cell is a transformed cell.

4. The method of claim 1, wherein the embryonic stem cell is a cell transformed to overexpress Oct3/4, Nanog, or Sox-2 protein as an embryonic stem cell-specific protein.

5. The method of claim 1, wherein the embryonic stem cell is a cell transformed to express at least one selected from the group consisting of a cell adhesion molecule, an antibody, a targeting protein, a cell membrane fusion protein, and a fusion protein thereof.

6. The method of claim 1, wherein a membrane of the microvesicle further includes a component other than a cell membrane of the embryonic stem cell.

7. The method of claim 6, wherein the component other than the cell membrane is cyclodextrin or polyethyleneglycol.

8. The method of claim 1, wherein the membrane component of the microvesicle is chemically modified.

9. The method of claim 1, wherein the composition further includes a component other than the embryonic stem cell-derived microvesicle.

10. The method of claim 9, wherein the component other than the embryonic stem cell-derived microvesicle is brefeldin A (BFA), BiP inducer X (BIX), or valproic acid (VPA).

11. An induced pluripotent stem cell prepared by the method any one of claim 1.

12. A cell therapy product comprising the induced pluripotent stem cell of claim 11.
